(19) United States
(12) Patent Application Publication
(10) Pub. No.: US 2022/0025314 A1
(43) Pub. Date: Jan. 27, 2022

(54) SIEVE SYSTEM AND METHODS FOR CELL MEDIA EXCHANGE

(71) Applicant: THE REGENTS OF THE UNIVERSITY OF CALIFORNIA, Oakland, CA (US)

(72) Inventor: Lawrence Kulinsky, Irvine, CA (US)

(21) Appl. No.: 17/450,494

(22) Filed: Oct. 11, 2021

Related U.S. Application Data
(63) Continuation-in-part of application No. 15/719,180, filed on Sep. 28, 2017, now Pat. No. 11,142,738.
(60) Provisional application No. 62/412,722, filed on Oct. 25, 2016.

(51) Int. Cl. C12M 1/12; C12M 1/00

(52) U.S. Cl. CPC C12M 25/04; C12M 29/04; C12M 23/50

(57) ABSTRACT

Methods and systems for cell media exchange wherein media may be aspirated without also aspirating cells and wherein removal and re-plating of cells is not necessarily required. For example, the cell culture system or apparatus features a gap that is small enough to retain cells therein and also sized to prevent media from leaking. The methods and systems of the present invention may help reduce stress and damage to cells as compared to traditional methods that require removing and re-plating cells.

### CROSS-REFERENCES TO RELATED APPLICATIONS

This application is a continuation-in-part and claims benefit of U.S. patent application Ser. No. 15/719,180, filed Sep. 28, 2017, which is a non-provisional and claims benefit of U.S. Provisional Patent Application No. 62/412,722 filed Oct. 25, 2016, the specifications of which are incorporated herein in their entirety by reference.

### FIELD OF THE INVENTION

The present invention relates to devices and methods for changing media for cells in cell culture, more particularly to methods and systems featuring a cell culture dish that allows for aspiration of media without contact with the cells in culture.

### BACKGROUND OF THE INVENTION

In order to grow cells of all types, including embryos, media that contains nutrients and growth factors is usually regularly changed, e.g., the old media is removed and new media (e.g., new type of media, same type of media) replaces the old. Typically, media exchange involves pipetting cells out of the dish and placing them in a new dish with the new media. This process of cell handling is stressful for cells, and it is always possible to damage the cells during the transfer. Alternatively, media may be aspirated and new media may be added. With this method, there is always a danger of aspirating the cells along with the media.

The present invention features methods and systems for cell media exchange, wherein media may be safely aspirated from the dish. For example, the present invention features a cell culture apparatus (e.g., cell culture dish, well, etc.) wherein media can be aspirated therefrom via a gap or slot that is small enough to retain cells therein and also sized to prevent media from leaking (e.g., via surface tension). Since media can be aspirated from the gap, there is typically not a need to handle the cells in a similar fashion as described above for previous methods; thus, the methods and systems of the present invention can help reduce stress and damage to cells.

### SUMMARY OF THE INVENTION

The present invention features a cell culture sieve system that allows media exchange without having to remove the cells. In some embodiments, the cell culture sieve system comprises a cell culture container adapted to grow or sustain cells in culture, wherein a gap is disposed on a side wall of the container. The gap is sized and adapted to retain media in the container in the absence of suction and to allow aspiration of the media in the presence of suction.

For example, the present invention features a cell culture sieve system comprising a well atop a culture substrate forming a container for holding media and cells in culture, wherein at least one gap is disposed on a side wall of the well, the at least gap is sized to retain media in the well when suction is not applied to the gap and to allow aspiration of said media from the well when suction is applied to the gap. The gap is sized to retain cells in the well when suction is applied to the gap.

The size of the gap may be chosen based on the size of the cells to be contained. For example, the gap may be smaller than the size of the cells. Surface tension may help prevent media from leaking through the gap. In some embodiments, the gap is from 0.05 microns to 100 microns at its largest dimension. In some embodiments, the gap is 0.05, 0.1, 1, 10, 20, 30, 40, 50, 60, 70, 80, 90, or 100 microns at its largest dimension. In some embodiments, the gap is from 0.05 to 10 microns, 10 to 50 microns, or 50 to 100 microns at its largest dimension. In some embodiments, the gap is greater than 100 microns at its largest dimension. (In some embodiments, the largest dimension is diameter.)

In some embodiments, the system further comprises an outer wall surrounding the well and spaced a distance apart from the well. In some embodiments, the system further comprises a pipette positioning area adjacent to a gap. An unobstructed pipette positioning area allows for alignment of a pipette tip with the gap such that the pipette tip may seal against the side wall. In some embodiments, the culture substrate is a part of a slide or plate. In some embodiments, the cell culture sieve system is part of a multi-well cell culture plate. In some embodiments, the side wall that forms the well is attached or connected to the culture substrate. In some embodiments, the slot is a cut or an indentation in the culture substrate.

In some embodiments, the system comprises a slot disposed in the culture substrate intersecting with the gap and fluidly connected to the gap. In some embodiments, the slot is a cut or an indentation connecting the well to the culture substrate.

In some embodiments, the system comprises a plurality of gaps extending from at or near a bottom edge of the well and upwardly toward a top edge (e.g., to the top edge, to the middle, to a place near the top edge, etc.) of the well.

The present invention also features a method of aspirating cell culture media (or a method of changing cell culture media). In some embodiments, the method comprises applying suction to a gap in a cell culture sieve system of the present invention, wherein suction applied to the gap removes media but not cells from the well of the cell culture sieve system. In some embodiments, applying suction to the gap comprises using a pipette to aspirate media through the gap. In some embodiments, the method is manual. In some embodiments, the method is automated. In some embodiments, the method further comprises adding new media to the well of the cell culture sieve system.

Without wishing to limit the present invention to any theory or mechanism, it is believed that the methods and systems of the present invention are advantageous because they allow the exchange of media in the same cell reservoir yet there is little or no aspiration of cells during the aspiration process. Further, the media exchange process can be accomplished without physically contacting the cells, thereby avoiding the physical stress associated with re-plating of cells. Surface tension may prevent the media from leaking from the wells through the gaps. When suction is applied to the gaps, the suction breaks the surface tension of the media and allows the media to be aspirated from the well.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1A shows a perspective view of a sieve system of the present invention comprising several gaps (the gaps are for media aspiration). The present invention is not limited to the configuration shown in FIG. 1A.

FIG. 1B shows a top view of a sieve system of the present invention wherein slots are present between the bottom of the well and the cell culture substrate (the slots are for media aspiration). The present invention is not limited to the configuration shown in FIG. 1B.

FIG. 1C shows a perspective view of a sieve system of the present invention comprising several gaps along the bottom edge of the well (the gaps are for media aspiration). The present invention is not limited to the configuration shown in FIG. 1C.

FIG. 2A shows a perspective view of a sieve system of the present invention. Gaps are disposed on a side wall of the well, and a slot is present between the bottom of the well and the cell culture substrate. The present invention is not limited to the configuration shown in FIG. 2A.

FIG. 2B shows a cross sectional view of the system of FIG. 2A. Note the present invention is not limited to the configuration shown in FIG. 2B.

FIG. 2C shows another cross sectional view of the system in FIG. 2A with a pipette positioned with its tip fluidly coupled with the slot at the bottom of the well so as to aspirate media from the well. The angle between the pipette and the side wall is selected such that a seal is formed between the pipette tip and the cell culture substrate.

FIGS. 3A-3B show a non-limiting example of aligning a pipette tip with a gap disposed between a top and bottom end of the side wall of the container to remove media from the container. In this example, a volume of the media is lowered to the level of the gap, but not completely removed from the container.

FIGS. 4A-4B show a non-limiting example of aligning a pipette tip with a bottom gap to completely remove media from the container.

FIGS. 5A-5C show different embodiments for aligning a pipette tip with the gap. FIG. 5A shows a pipette tip aligned with a gap. FIG. 5B shows a pipette tip with a rubber attachment disposed at the end. The rubber attachment allows the pipette tip to form a better seal with the gap compared to a pipette tip with no rubber attachment. FIG. 5C shows an aspiration socket disposed in the culture substrate to allow for better alignment of the pipette tip with the gap.

FIGS. 6A-6D show non-limiting examples of the gap disposed on the side wall. The gap may be a single hole or a plurality of holes disposed on the side wall. The plurality of holes may form a gap pattern, where the gaps may be different shapes and/or sizes.

FIGS. 7A-7B show embodiments of the cell culture sieve system being a part of a multi-well plate. FIG. 7A shows a top view of the multi-well plate that illustrates the unobstructed pipette positioning areas (dotted lines). The horizontal and vertical spacing between the wells allow the pipette tip to access the gaps via the resulting unobstructed pipette positioning areas. FIG. 7B shows a side view of the multiwell plate.

FIGS. 8A-8B show how the pipette may be positioned vertically in the pipette positioning area. The vertical angular range of the pipette positioning area allows the pipette to be positioned in perfect alignment with a pipette positioning axis or at a angle vertically offset from the pipette positioning axis.

FIGS. 9A-9B show how the pipette may be positioned horizontally in the pipette positioning area. The horizontal angular range of the pipette positioning area allows the pipette to be positioned in perfect alignment with a pipette positioning axis or at a angle horizontally offset from the pipette positioning axis.

FIG. 10 shows an example of a pipette with an L-shaped tip. This type of pipette allows for alignment of the pipette tip with the gap while the pipette is upright and perpendicular to the axis of the gap.

FIG. 11 shows a side view of the container with gaps and aspiration sockets disposed on the side wall.

### DETAILED DESCRIPTION OF THE INVENTION

Following is a list of elements corresponding to a particular element referred to herein:
100 sieve system
102 media (e.g., with cells)
105 cell culture substrate (e.g., plate, etc.)
110 well
122 gap
128 slot
130 pipette
132 pipette tip
134 aspiration socket
136 pipette positioning area
138 rubber attachment

The present invention cell culture sieve systems and methods for media exchange featuring a cell culture dish that allows for aspiration of media without contact with the cells in culture.

The present invention features a cell culture sieve system (100). The cell culture sieve system may comprise: a) a culture substrate (105); b) a side wall (110) disposed atop the culture substrate (105) to form a container for holding media and cells in culture; c) a gap (122) disposed in the side wall (110) to directly connect an interior of the container with an exterior of the cell culture sieve system; and d) a slot (128) disposed between a bottom of the side wall (110) and the culture substrate (105) such that the slot (128) is fluidly connected with the gap (122). In some embodiments, the gap (122) is configured to retain media via surface tension and to prevent media from leaking from the container. In other embodiments, the slot (128) is sized and configured to receive a pipette tip (132). Without wishing to limit the present invention to any theory or mechanism, when suction is applied to the gap (122) via a pipette tip (132) positioned within the gap (122) media is aspirated from the container.

In some embodiments, the cell culture sieve system (100) may comprise: a) a culture substrate (105); b) a side wall (110) disposed atop the culture substrate (105) to form a container for holding media and cells in culture; c) a gap (122) disposed in the side wall (110) to directly connect an interior of the container with an exterior of the cell culture sieve system; and d) an unobstructed pipette positioning area (136), such that a pipette (130) may be positioned with its tip (132) in contact with the side wall (110) at an angle such that the tip (132) and the side wall (110) form a seal. In some embodiments, the cell culture sieve system (100) may further comprise a pipette (130) within the pipette positioning area (136), positioned with its tip (132) in contact with the side wall (110) at an angle such that the tip (132) and the side wall (110) form a seal.

As used herein, the term “pipette positioning area” refers to a three-dimensional area adjacent to a gap (122) in a side wall (110) where a pipette may be positioned such that the pipette is fluidly coupled with the gap (122) and the pipette tip (132) seals against the side wall (110). The pipette positioning area (136) may surround a pipette positioning axis, along which the pipette is positioned for best alignment with the gap (122). As a non-limiting example, the pipette positioning axis may be orthogonal to the surface of the side wall (110). A radius of the pipette positioning area (136) may be about equal to or slightly larger than the length of a pipette. In some embodiments, the pipette positioning area (136) may be cone-shaped, with boundaries offset by an angle from a pipette positioning axis. In some embodiments, the pipette positioning area (136) may be defined by a radius, a vertical angle, and a horizontal angle. In preferred embodiments, the pipette positioning area (136) is unobstructed, such that a pipette may be positioned along the pipette positioning axis, with the tip (132) in contact with the side wall (110).

The cell culture sieve system (100) of the present invention may comprise: a) a culture substrate (105); b) a side wall (110) disposed atop the culture substrate (105) to form a container for holding media and cells in culture; c) a gap (122) disposed in the side wall (110) to directly connect an interior of the container with an exterior of the cell culture sieve system; and d) an aspiration socket (134) disposed in the side wall (110) around the gap so as to guide positioning of a pipette tip (132) to form a seal between the tip (132) and the side wall (110) such that media may be aspirated through the gap (122) via the pipette tip (132).

The present invention also features a cell culture sieve system (100) comprising: a) a culture substrate (105); b) a side wall (110) disposed atop the culture substrate (105) to form a container for holding media and cells in culture; and c) at least one gap (122) disposed on the side wall (110). In some embodiments, the at least one gap (122) is configured to retain media via surface tension and to prevent media from leaking from the container. Without wishing to limit the present invention to any theory or mechanism, when suction is applied to the at least one gap (122), media is aspirated from the container via the at least one gap (122). In other embodiments, the at least one gap (122) is sized and configured to prevent cells within the container from exiting the container.

The present invention features a cell culture sieve system (100) comprising: a) a well (110) atop a culture substrate (105); and b) at least one gap (122) directly coupled to the single side wall of the well (110) between a bottom edge of the well and a top edge of the well. In preferred embodiments, the at least one gap (122) prevents media from leaking from the well (110) when suction is not applied to the gap (122), and when suction is applied to the at least one gap (122), the at least one gap (122) allows aspiration of said media directly from the well (110) but is sized to prevent cells from being suctioned out of the well (110). In some embodiments, the well (110) forms a container for holding media and cells in culture. In other embodiments, the well (110) comprises a single side wall.

The present invention also features a cell culture sieve system (100) comprising: a) a culture substrate (105); b) a well (110) atop the culture substrate (105); c) at least one gap (122) directly coupled to the single side wall of the well (110) between a bottom edge of the well and a top edge of the well; and d) a pipette. Without wishing to limit the present invention to any theory or mechanism, when the pipette is aligned with the at least one gap (122), suction is applied to the at least one gap (122) via the pipette (130), allowing for aspiration of media directly from the well (110). In some embodiments, the well (110) forms a container for holding media and cells in culture. In other embodiments, the well (110) comprises a single side wall.

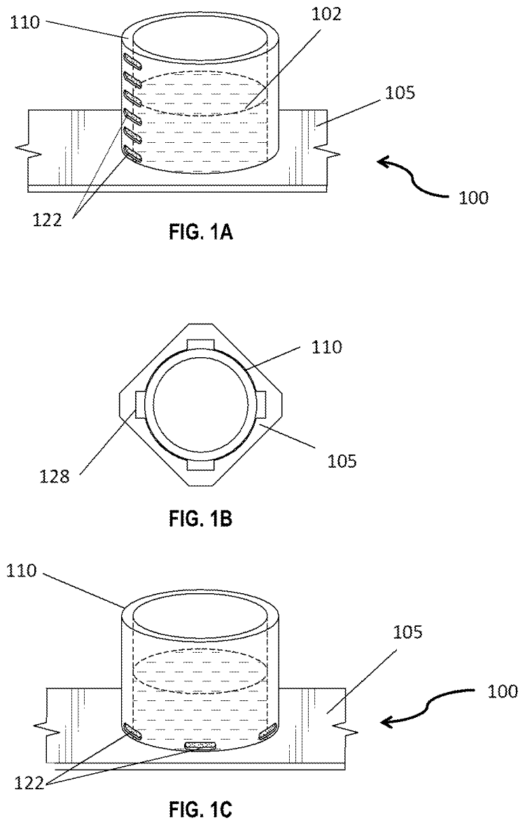

Referring to FIG. 1A, FIG. 1B, and FIG. 1C, the sieve system (100) of the present invention comprises a well (110) that forms a container to hold media (102). The media may have cells in culture. The well (110) may be a part of a cell culture substrate (105). In some embodiments, the cell culture substrate may be a part of any appropriate cell culture mechanism. As non-limiting examples, the cell culture substrate (105) may be a part of a slide or a plate. In other embodiments, the cell culture sieve system (100) may be a single dish, a part of a multi-well plate, or any other appropriate cell culture mechanism. Note in FIG. 1A and FIG. 1C, the culture substrate (105) resembles a plate or a strip. The present invention is not limited to this configuration.

In some embodiments, the gap (122) is a hole disposed in the side wall (110). In other embodiments, the gap (122) may be disposed between a bottom edge of the container and a top edge of the container. In preferred embodiments, the gap (122) prevents media from leaking from the container via surface tension. In one embodiment, the gap (122) prevents media from leaking from the container when suction is not applied to the gap (122), and when suction is applied to the gap (122), the gap (122) allows aspiration of said media directly from the container.

In other embodiments, the gap (122) is directly connected to an exterior of the cell culture system. In other embodiments, the gap (122) may provide size selection. For example, the gap (122) may be sized to retain cells in the container when media is aspirated from the container. Thus, a gap size may be chosen based on the size of a cell of interest.

As a non-limiting example, if the cell of interest were approximately 100 microns in diameter, a gap of less than 100 microns (e.g., 70 microns) would be selected. If the cell was 10 microns in diameter, a gap of less than 10 microns (e.g., 5 microns) may be chosen.

In some embodiments, the gap (122) is sized between about 0.5 microns to 100 microns at its largest dimension (e.g., diameter). In some embodiments, the gap (122) is sized to be about 0.05 microns, 1 micron, 5 microns, 10 microns, 20 microns, 30 microns, 40 microns, 50 microns, 60 microns, 70 microns, 80 microns, 90 microns, or 100 microns at its largest dimension (e.g., diameter). In some embodiments, the gap (122) is sized between about 0.05 to 10 microns at its largest dimension (e.g., diameter). In some embodiments, the gap (122) is sized between about 1 to 10 microns at its largest dimension (e.g., diameter). In some embodiments, the gap (122) is sized between about 10 to 50 microns at its largest dimension (e.g., diameter). In some embodiments, the gap (122) is sized between about 50 to 100 microns (e.g., 70-75 microns) at its largest dimension (e.g., diameter). In some embodiments, the gap (122) is smaller than about 100 microns at its largest dimension (e.g., diameter). In some embodiments, the gap (122) is larger than about 100 microns at its largest dimension (e.g., diameter).

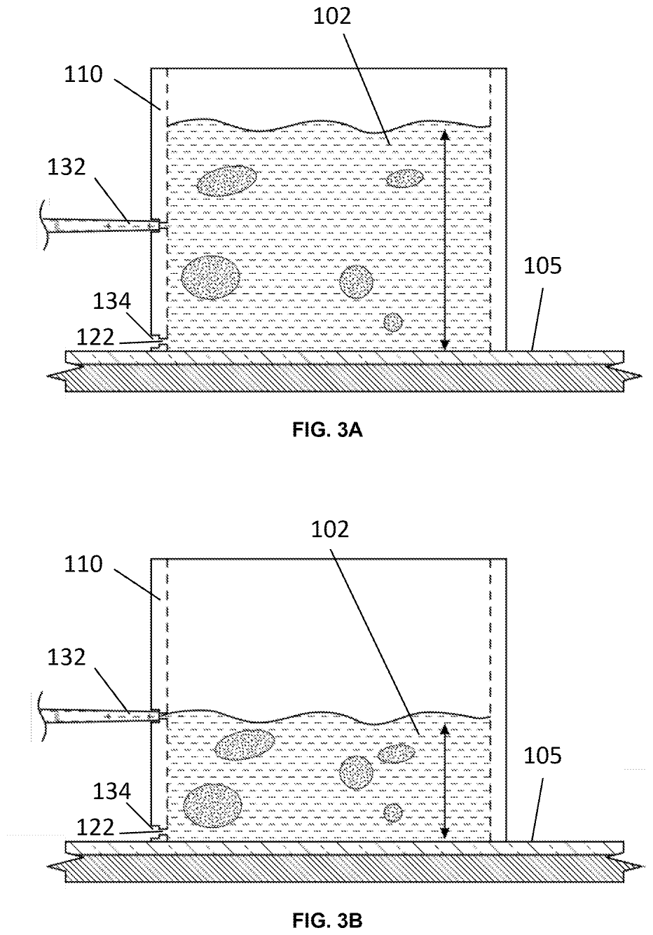

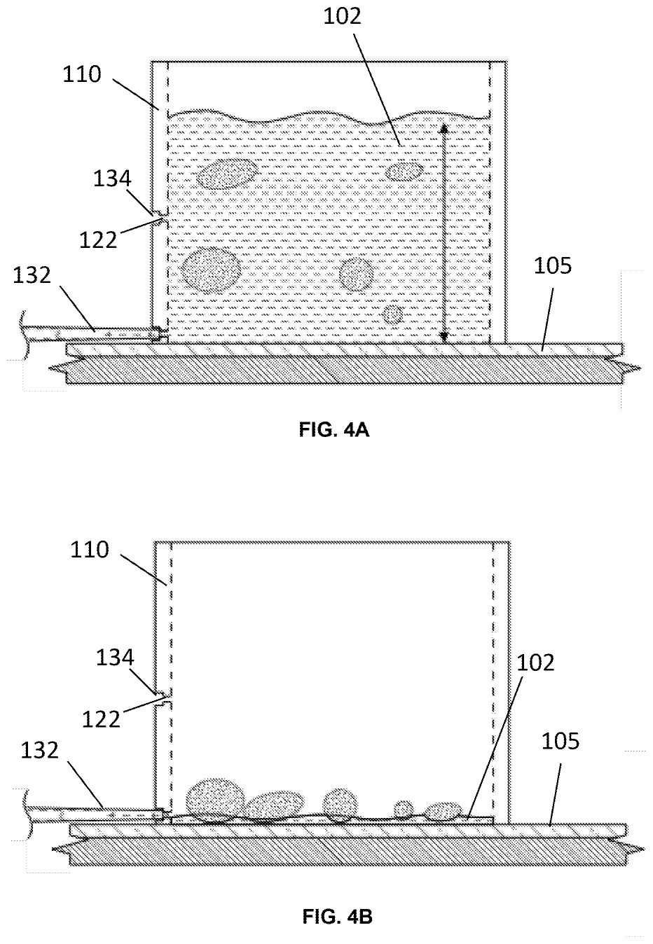

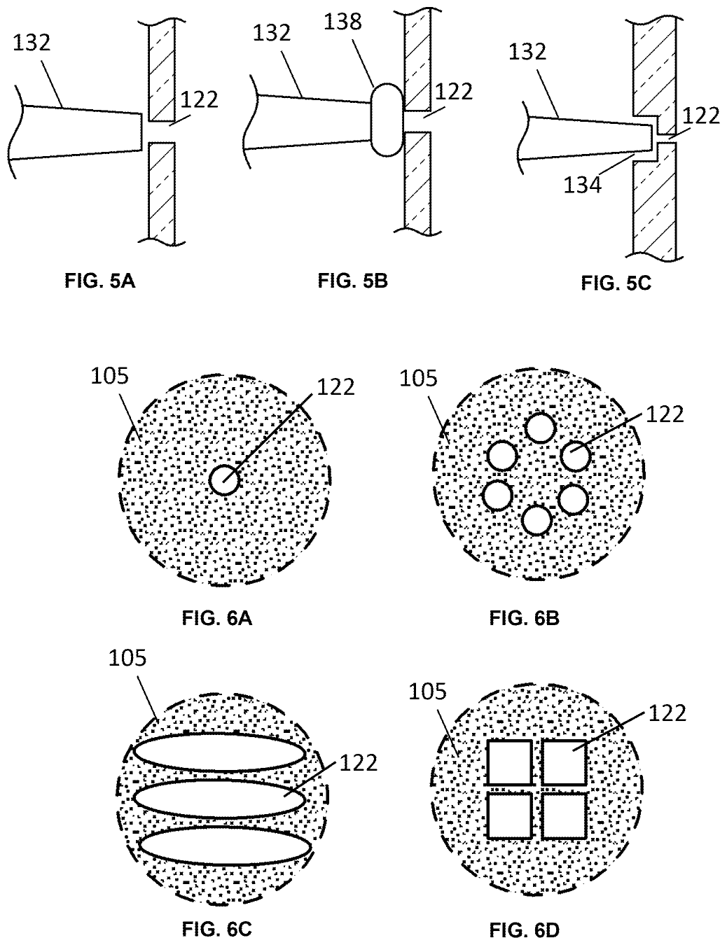

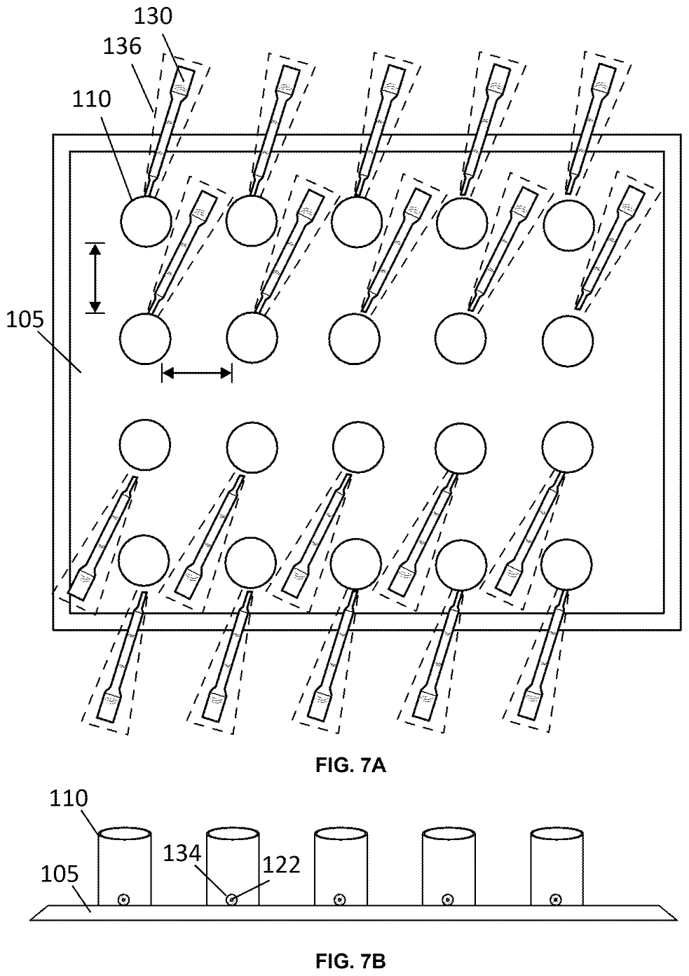

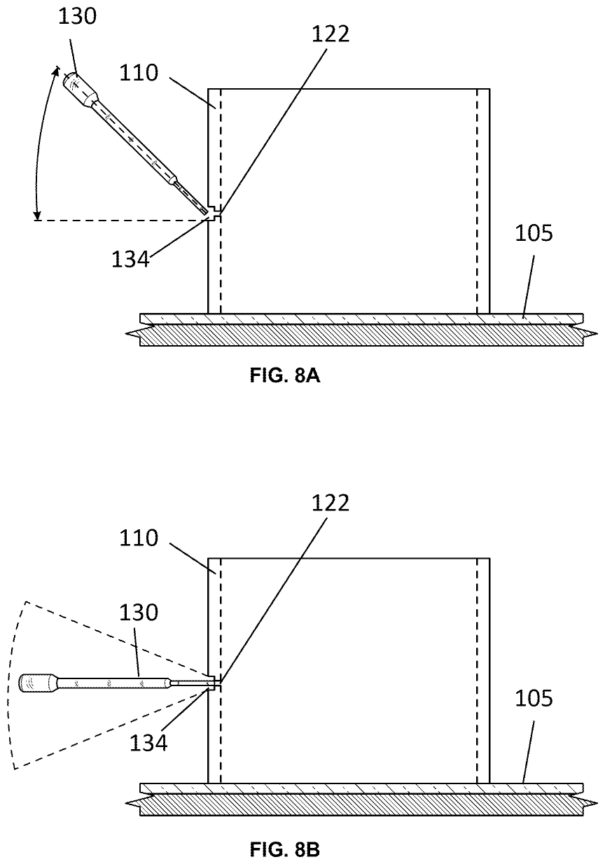

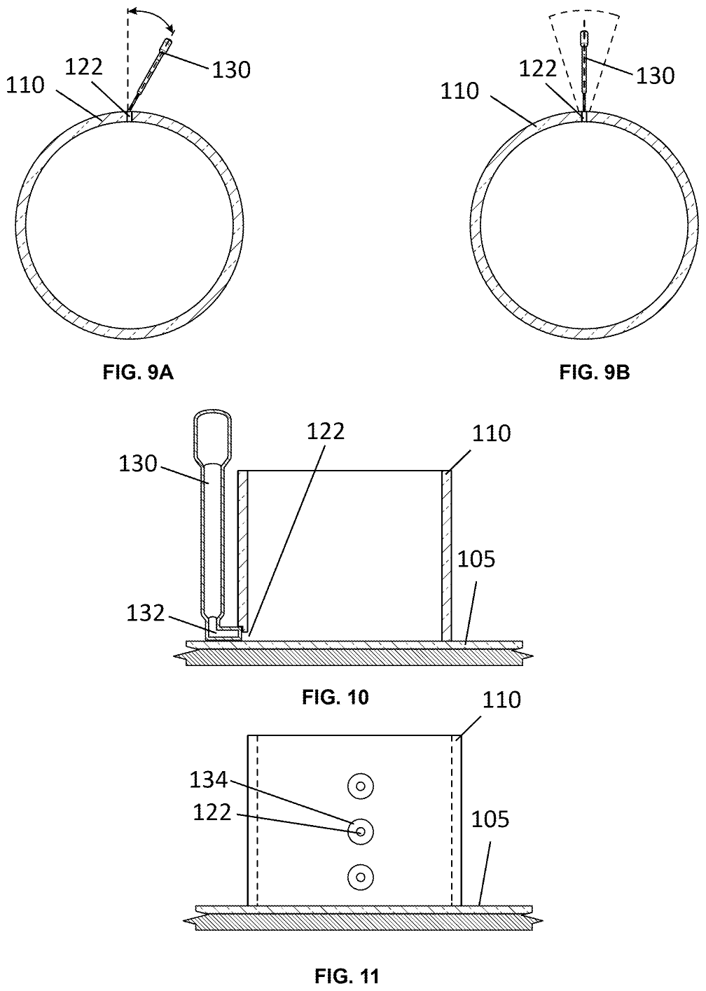

In yet another embodiment, the cell culture sieve system comprises a plurality of gaps (122) extending from at or near a bottom edge of the container upwardly toward a top edge of the container. For example, as shown in FIGS. 1A, 2A-2C, 3A-3B, and 4A-4B, gaps (122) are disposed at the intersection of the bottom of the well (110) and the cell culture substrate (105), as well as in locations higher up toward the top of the well (110). A user may choose a gap from which media is to be aspirated. FIG. 1C shows several gaps (122) along the bottom edge of the well (110). In this embodiment, a user may use one gap of the plurality of gaps to completely empty the well of media. In the embodiment shown in FIGS. 3A-3B, a user may use one gap of the plurality of gaps to reduce the amount of media in the well without completely emptying the well.

In other embodiments, the cell culture sieve system of the present invention comprises a plurality of gaps (122) disposed in the side wall. In one embodiment, the plurality of gaps (122) are disposed between a top edge and a bottom edge of the side wall. In some embodiments, each gap in the plurality of gaps is centrally aligned on a same vertical axis with one another. In another embodiment, each gap in the plurality of gaps are centrally aligned on a same horizontal axis with one another. In yet another embodiment, the plurality of gaps form a gap pattern. The gap pattern may be a plurality of gaps (122) concentrated in one area of the side wall. In some embodiments, each gap in the plurality of gaps is the same size. In other embodiments, each gap in the plurality of gaps is a different size. In further embodiments, each gap in the plurality of gaps is circular, ovular, rectangular, square-shaped or a combination thereof. In yet another embodiment, the gap (122) may be disposed at a bottom edge of the container, such that all of the media may be aspirated from the container. In some embodiments, the gap (122) is disposed between a bottom edge and a top edge of the side wall. In this configuration, media is aspirated from the gap (122) to lower a volume of media in the container without completely emptying the container.

In some embodiments, the cell culture sieve system (100) is a part of a multi-well plate. In other embodiments, the side wall is a single side wall. The single side wall may be one wall that forms the container. The single side wall may form a barrier between the media in the container and the exterior of the container. In this embodiment, the container is independent from any other containers that may be disposed on the culture substrate. Each container has its own side wall such that each container is independent from other containers of the multi-well plate. The container is not fluidly connected to other containers on the culture substrate. The gap disposed on the side wall connects an interior of the container to an exterior of the container such that when media is in the container, the media is in contact with air through the gap.

In some embodiments, an exterior of the container is not configured to contain fluid. In other embodiments, the cell culture sieve system does not have an exterior wall. A pipette may be able to access the gap from a top or from a side of the system. In yet another embodiment, a mark may be disposed on the side wall to indicate where the gap is located on the wall. The mark may help a user find the gap to aspirate media from the container.

In preferred embodiments, a pipette is able to access the gap (122) with no obstructions. The unobstructed pipette positioning area may allow positioning of a pipette such that its tip (132) is in contact with the side wall (110) at an angle such that the tip (132) and the side wall (110) form a seal. The pipette positioning area (136) may also be a spacing around the container that allows a pipette to access the gap (122). In a multi-well plate, the pipette positioning area (136) may be an unobstructed area between the containers on the plate that allows the pipette to access the gap (122). In the pipette positioning area (136), a micropipette may be positioned at an angle between about 0° to 90° from the line from a top of the container to the gap (122). A transfer pipette may have access to the gap (122) at an angle between about 0° to 180°, from the line from a top of the container to the bottom of the container.

In some embodiments, the gap (122) may be configured to receive a pipette tip (132) for suctioning media from the container. In the configuration where the system is a part of a multi-well plate, the containers may be spaced to allow a pipette tip (132) to access the one gap (122). The pipette tip (132) may interact with the one gap (122) at an angle (θ) between about 0° to 90°. In some embodiments, the pipette tip (132) may interact with the gap (122) at an angle between about 0° to 10°, 10° to 20°, 20° to 30°, 30° to 40°, 40° to 50°, 50° to 60°, 60° to 70°, 70° to 80°, or 80° to 90°.

In some embodiments, the tip (132) of the pipette forms a seal with the gap (122). The tip (132) of the pipette may comprise a flexible material that forms a seal with the gap (122). As a non-limiting example, the pipette tip (132) may have a rubber attachment (138) at the end to form a seal with the gap (122). In another embodiment, a flexible material may be disposed around the gap (122) to form a seal with a pipette tip (132). In yet another embodiment, the pipette tip (132) may be L-shaped.

The size of the gap (122) may require different pipette shapes or materials in order to create a seal to suction the media out of the well. If the gap (122) is large, this does not require a perfect seal because it takes less suction to break the surface tension of the media. A large gap (122) may be about 50 microns or larger. If the gap (122) is small, a pipette tip (132) that is pliable may be needed in order to make a better seal with the gap (122) to suction out the media from the well. A small gap (122) may be sized between about 0.5 microns to 50 microns. As a non-limiting example, the pipette tip (132) may be made out of rubber to create a seal with the gap (122). Alternatively, to create a better seal with the gap (122), an indentation can be molded into the side wall around the gap (122) such that the pipette tip (132) fits into the indentation. In some embodiments, the pipette tip (132) may be L-shaped to contact the indentation. An L-shaped pipette tip (132) may allow a user to align the pipette tip (132) orthogonal to the well to suction out the media from the well. This type of pipette tip (132) may be useful when the well is a part of a multi-well plate where access to gaps in wells in the middle of the multi-well plate may be obstructed by neighboring wells.

The cell culture system of the present invention may further comprise a slot fluidly connected to the gap (122). In some embodiments, the slot is a cut or an indentation in the culture substrate. In one embodiment, the slot is an indentation in the culture substrate that surrounds the gap (122). The slot may be used to guide a pipette tip (132) to the gap (122). A pipette tip (132) may fit into the slot. In preferred embodiments, the slot is configured to receive a pipette tip (132) for aspirating media from the gap (122).

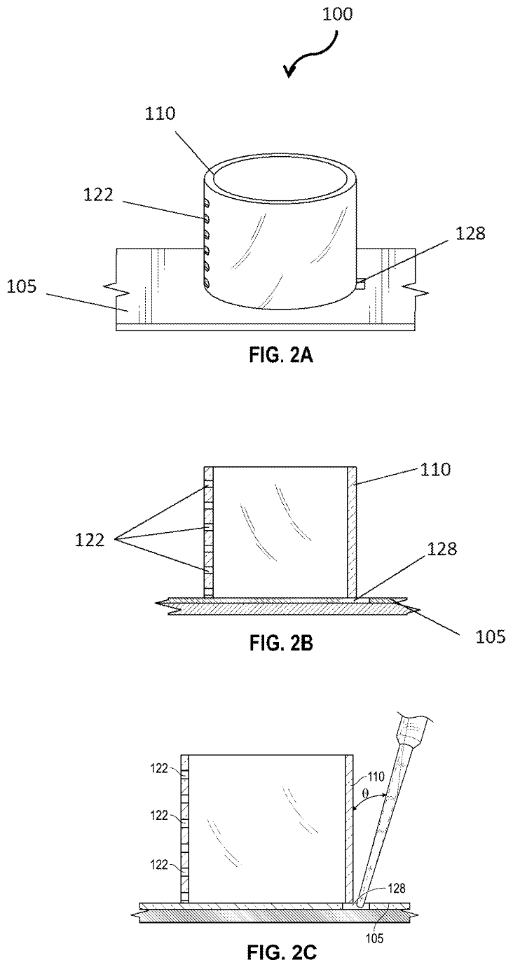

As shown in FIG. 1B, FIG. 2A and FIG. 2B, in some embodiments, the system (100) further comprises slots (128). In preferred embodiments, the slots (128) may be located at an intersection where the well (110) is attached to the cell culture substrate (105). The slots (128) are similar to the gaps (122) in terms of size and function. However, the gaps (122) are holes in the cell culture substrate (105) and pass all of the way through the side wall, whereas the slots (128) are cuts or indentations in the cell culture substrate (105) that do not pass all of the way through the side wall. In some embodiments, a slot (128) intersects with a gap (122) and is fluidly connected to the gap (122). In other embodiments, the slot (128) allows for aligning a pipette tip (132) with the gap (122). In the configuration where the container is a part of a multi-well plate, the containers may be spaced to allow a pipette tip (132) to access the slots (128). The pipette tip (132) may interact with the slots (128) at an angle (θ) between about 0° to 45°. In some embodiments, the pipette tip (132) may interact with the slots at an angle between about 0° to 10°, 10° to 20°, 20° to 30°, 30 to 40°, or 40° to 45°. The slots (128) may allow for aspiration of all of the media disposed in the well.

In further embodiments, the cell culture sieve system of the present invention comprises an aspiration socket (134). The aspiration socket (134) may be an indentation in the cell culture substrate (105) or in the side wall (110). In other embodiments, the aspiration socket (134) may be formed via a protrusion from the cell culture substrate (105) or the side wall (110). In some embodiments, the aspiration socket (134) is configured to receive a pipette tip (132) for aspirating media from the gap (122). The aspiration socket (134) may be a slot (128). In other embodiments, the aspiration socket (134) is fluidly connected to the gap (122). In one embodiment, an interior diameter of the aspiration socket (134) is sized to seal around an exterior diameter of the pipette tip (132).

In other embodiments, the container is filled with media (102) using an opening at a top end of the well. The gaps (122) may not be used to fill the container with media (102). In some embodiments, the gap (122) directly connects the container with an exterior of the cell culture system. As a non-limiting example, direct connection of the interior of the container with the exterior of the cell culture system via a gap (122) means that fluid which passes from the container through the gap would no longer be contained by the cell culture system. The container may only have a single side wall (110); with no additional walls surrounding the container such that the container is fluidly connected to another container or any other vessel or chamber that may be used to hold media. The container is independent from any other containers that may be disposed on the culture substrate. In this configuration, the gap (122) forms an interface between the media (102) and air.

In some embodiments, the container is mounted, attached or connected to the cell culture substrate (105). As a non-limiting example, the container may be connected to the cell culture substrate (105) via an adhesive, e.g., a double-sided adhesive. The present invention is not limited to this construction. For example, the container may be constructed as a single unit (e.g., via injection molding or other processes), e.g., the well has a base floor or is directly constructed on the cell culture dish).

In some embodiments, an inner wall is disposed in the container (not shown), e.g., an inner wall forming an enclosure. The inner wall may be very short in height, e.g., about the height of a cell (e.g., 10-15 um, 15-20 um, 20-30 um, etc. The inner wall may be for helping to enclose the cells or sequester the cells in a particular area on the cell culture substrate (105) within the container.

The present invention described herein uses specially designed cell culture wells or containers (e.g., wells, beakers, etc.) that allow for media exchange to take place with cells remaining in the same container and where these cells are not contacted during this media exchange. Surface tension keeps media from leaking from the gaps (122) of the systems described herein. In some embodiments, when the media needs to be exchanged, a pipette tip (132) is placed outside the gap (122) and suction is applied to the gap (122) to aspirate the media from the well. The suction that is applied to the gap (122) breaks the surface tension of the media and allows the media to be aspirated from the well. Once the media is aspirated, new media may be added to the well from the top of the well. In other embodiments, the pipette tip (132) may be aligned with the gap (122) using a slot or an aspiration socket. In this embodiment, the pipette tip (132) is aligned with the gap prior to aspiration of the media.

In one embodiment, a double sided adhesive is used to connect the well to the cell culture substrate. In some embodiments, the gap (122) is disposed in the double-sided adhesive (120). The present invention is not limited to the user of double sided adhesive. For example, the gap (122) may be disposed in a side wall of the well or in other components of the system. The suction force is stronger than the capillary surface tension holding the media in the well and the media can be evacuated, while the cells stay in the well due to size exclusion principle, as their size is larger than the gap size.

The system of the present invention may feature a slightly hydrophobic surface that will not allow fluid (e.g., media) to escape through the gap. For example, in some embodiments, the material used to form the system or components thereof (e.g., the gap) may be chosen to be slightly hydrophobic (e.g., a plastic). In some embodiments, the material is hydrophilic but is coated (or a portion is coated, e.g., the gap) to be slightly hydrophobic. In some embodiments, the gap may be slightly hydrophilic so as to allow fluid to go through but only to form a droplet just outside of the gap (so fluid doesn't escape further).

In some embodiments, the media exchange is manual. In some embodiments, the media exchange is automated.

The present invention features methods for aspirating media from a cell culture sieve system without contacting the cells in culture. The method may comprise providing any of the cell culture sieve systems described herein, adding media and cells to the container, incubating the cells, aspirating the media, and replacing the media. To aspirate the media from the container, a pipette may be used to suction the media from the container. In some embodiments, the pipette may be aligned with the gap to form a seal with the gap to suction the media from the container. The gap of the cell culture sieve system is sized such that media is retained in the container when suction is not applied to the gap, but when suction is applied to the gap, only media is aspirated and the cells stay in the container. In other embodiments, the pipette may be aligned with the obstructed pipette positioning area prior to aspirating the media.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A cell culture sieve system (100) comprising:
a. a culture substrate (105);
b. a side wall (110) disposed atop the culture substrate (105) to form a container for holding media and cells in culture;
c. a gap (122) disposed in the side wall (110) to directly connect an interior of the container with an exterior of the cell culture sieve system, wherein the gap (122) is configured to retain media via surface tension and to prevent media from leaking from the container; and
d. a slot (128) disposed between a bottom of the side wall (110) and the culture substrate (105) such that the slot (128) is fluidly connected with the gap (122), wherein the slot (128) is sized and configured to receive a pipette tip (132);
wherein when suction is applied to the gap (122) via a pipette tip (132) positioned within the gap (122) media is aspirated from the container.

2. A cell culture sieve system (100) comprising:
a. a culture substrate (105);
b. a side wall (110) disposed atop the culture substrate (105) to form a container for holding media and cells in culture;
c. a gap (122) disposed in the side wall (110) to directly connect an interior of the container with an exterior of the cell culture sieve system; and
d. an unobstructed pipette positioning area (136), such that a pipette (130) may be positioned with its tip (132) in contact with the side wall (110) at an angle such that the tip (132) and the side wall (110) form a seal.

3. The system (100) of claim 2, additionally comprising a pipette (130) within the pipette positioning area (136), positioned with its tip (132) in contact with the side wall (110) at an angle such that the tip (132) and the side wall (110) form a seal.

4. The system (100) of claim 2, further comprising a plurality of gaps (122) disposed in the side wall.

5. The system (100) of claim 4, wherein the plurality of gaps are disposed between a top edge and a bottom edge of the side wall.

6. The system of claim 4, wherein the plurality of gaps form a gap pattern.

7. The system of claim 4, wherein each gap in the plurality of gaps is circular, ovular, rectangular, square-shaped or a combination thereof.

8. The system of claim 2 wherein the gap (122) prevents media from leaking from the container via surface tension.

9. The system of claim 2, wherein the gap (122) is sized to retain cells when media is aspirated from the container.

10. The system of claim 2, wherein the gap (122) is directly connected to an exterior of the cell culture system.

11. The system of claim 2, wherein the wall is a single side wall.

12. The system of claim 2, further comprising a slot (128) fluidly connected to the gap (122).

13. The system of claim 12, wherein the slot (128) is configured to receive a pipette tip (132) for aspirating media from the gap (122).

14. The system of claim 12, wherein the slot (128) is a cut or an indentation in the culture substrate (105).

15. The system of claim 3, wherein the tip (132) of the pipette (130) forms a seal with the gap (122).

16. The system of claim 3, wherein the pipette tip (132) is L-shaped.

17. The system of claim 3, wherein a flexible material is disposed at an end of the pipette tip (132).

18. A cell culture sieve system (100) comprising:
a. a culture substrate (105);
b. a side wall (110) disposed atop the culture substrate (105) to form a container for holding media and cells in culture;
c. a gap (122) disposed in the side wall (110) to directly connect an interior of the container with an exterior of the cell culture sieve system; and
d. an aspiration socket (134) disposed in the side wall (110) around the gap so as to guide positioning of a pipette tip (132) to form a seal between the tip (132) and the side wall (110) such that media may be aspirated through the gap (122) via the pipette tip (132).

19. The system (100) of claim 18, wherein an interior diameter of the aspiration socket (134) is sized to seal around an exterior diameter of the pipette tip (132).

20. The system (100) of claim 18, wherein the aspiration socket (134) is an indentation in the cell culture substrate (105).
